(19) United States
(12) Patent Application Publication
(10) Pub. No.: US 2026/0014334 A1
(43) Pub. Date: Jan. 15, 2026

(54) SENSOR DEVICE FOR VOLUMETRIC CAPNOGRAPHY

(71) Applicant: Masimo Corporation, Irvine, CA (US)

(72) Inventor: Björn Danwihl, Vallentuna (SE)

(21) Appl. No.: 19/258,125

(22) Filed: Jul. 2, 2025

Related U.S. Application Data
(60) Provisional application No. 63/669,637, filed on Jul. 10, 2024.
(60) Provisional application No. 63/669,644, filed on Jul. 10, 2024.

(51) Int. Cl. A61M 16/00; A61M 16/04

(52) U.S. Cl. CPC A61M 16/0003; A61M 16/0402; A61M 2205/0216; A61M 2205/0294; A61M 2205/3306; A61M 2205/3375; A61M 2205/583; A61M 2230/432

(57) ABSTRACT

Disclosed herein is a sensor device for volumetric capnography. The sensor device is coupled to an adapter comprising a housing that encloses a measurement volume. The adapter is configured for gas flow along a first length axis, which aligns with a second length axis of the sensor device. The device includes an IR light source and an IR light detector, positioned on opposite sides of the housing. The IR light source emits IR light toward the detector across the measurement volume. The sensor device also includes a first ultrasonic transceiver in contact with the adapter and configured to emit ultrasound centered at a first position along the second length axis, directed partly along the axis and partly transversely. A second ultrasonic transceiver, also in contact with the adapter, emits ultrasound centered at a second position along the second length axis, directed partly opposite to the first direction and partly transversely.

### RELATED APPLICATIONS

Any and all applications for which a foreign or domestic priority claim is identified in the Application Data Sheet as filed with the present application are hereby incorporated by reference under 37 CFR 1.57.

This application claims the priority benefit of U.S. Provisional Patent Application 63/669,637 filed on Jul. 10, 2024, entitled “SENSOR DEVICE FOR VOLUMETRIC CAPNOGRAPHY,” and U.S. Provisional Patent Application 63/669,644 filed on Jul. 10, 2024, entitled “ADAPTER FOR VOLUMETRIC CAPNOGRAPHY,” which are incorporated by reference herein in their entirety.

### BACKGROUND

### Field

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

The present disclosure relates to a sensor device and an adapter for volumetric capnography. The sensor device can be configured to be attached to an adapter comprising a housing that encloses a measurement volume, and enables simultaneous measurement of gas flow through the adapter and the concentration of a tracer substance in the gas. More specifically, the adapter can be designed to be positioned between a tube inserted into the respiratory tract of a patient and a tube connected to a ventilator.

### Related Art

Carbon dioxide (CO2) is the most abundant gas produced by the human body. It is well known that CO2 is the primary drive to breathe and a primary motivation for mechanically ventilating a patient. Monitoring the CO2 level during respiration (capnography) is noninvasive, easy to do, relatively inexpensive, and has been studied extensively. Over the last few decades, Capnography has improved. This is thanks to the development of faster infrared sensors that can measure CO2 at the airway opening in real time. By knowing how CO2 behaves on its way from the bloodstream through the alveoli to the ambient air, physicians can obtain useful information about ventilation and perfusion. However, also, other tracer substances may be of interest.

### SUMMARY

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these implementations are intended to be within the scope of the invention herein disclosed. These and other implementations will become readily apparent to those skilled in the art from the following detailed description of the preferred implementations having reference to the attached figures.

In some implementations, a sensor device for volumetric capnography configured to be attached to an adapter can include: a housing enclosing a measurement volume, wherein the adapter is configured for gas flow along a first length axis through the measurement volume, and wherein a second length axis of the sensor device is aligned with the first length axis; an infrared (IR) light source and an IR light detector, wherein the IR light source and the IR light detector are positioned on opposite sides of the housing, the IR light source configured to emit IR light in an emission direction towards the IR light detector, wherein the emission direction is at an angle to the second length axis, and wherein the second length axis and the emission direction define a symmetry plane; a first ultrasonic transceiver configured to contact the adapter and to emit an ultrasound centered at a first position along the second length axis, the ultrasound directed at least partly in a first direction along the second length axis and at least partly transversally to the second length axis; and a second ultrasonic transceiver configured to contact the adapter and to emit ultrasound centered at a second position along the second length axis, the ultrasound being directed at least partly in a second direction opposite to the first direction along the second length axis and at least partly transversal to the second length axis.

In some implementations, the first ultrasonic transceiver is configured to detect ultrasound that has been emitted from the second ultrasonic transceiver, and the second ultrasonic transceiver is configured to detect ultrasound that has been emitted from the first ultrasonic transceiver. In some implementations, the first ultrasonic transceiver and the second ultrasound transceiver are both arranged on a same side of the symmetry plane, and wherein the first ultrasonic transceiver is configured to detect ultrasound from the second ultrasonic transceiver, which ultrasound has travelled through the measurement volume and has been reflected in the adapter. In some implementations, the first ultrasonic transceiver is configured to emit ultrasound in a first main direction at a first angle (α1) to the second length axis, and the second ultrasonic transceiver is configured to emit ultrasound in a second main direction at a second angle (α2) to the second length axis, wherein a size of the first angle (α1) is equal to a size of the second angle (α2).

In some implementations, the IR light is emitted from a spot arranged between the first position and the second position along the second length axis. In some implementations, the emission direction of the IR light is perpendicular to the second length axis. In some implementations, at least one of the first ultrasonic transceiver is circularly cylindrical and the second ultrasonic transceiver is circularly cylindrical.

In some implementations, at least one of the first ultrasonic transceiver and the second ultrasonic transceiver includes: an acoustic impedance matching layer having an inner side and an outer side configured to be in contact with the adapter; a piezoelectric disk attached in contact with the inner side of the acoustic impedance matching layer; and a backing layer in contact with the piezoelectric disk, wherein the piezoelectric disk is positioned between the acoustic impedance matching layer and the backing layer. In some implementations, the acoustic impedance matching layer includes a porous material. In some implementations, the acoustic impedance matching layer includes an organic binder and silicon dioxide powder. In some implementations, the backing layer includes a resilient layer in contact with the piezoelectric disk.

In some implementations, the sensor device includes an RFID tag including unique identification data. In some implementations, the adapter includes: a first tube portion including a first opening in a first end of the first tube portion, wherein a second end of the first tube portion is coupled to a first end of the housing; a second tube portion including a second opening in a first end of the second tube portion, wherein a second end of the second tube portion is coupled to a second end of the housing; and at least a first transparent window and a second transparent window in the housing, the first transparent window and the second transparent window providing a line of sight through the measurement volume; wherein the adapter includes at least one membrane attached to the housing, wherein the at least one membrane separates the measurement volume from an outside of the adapter, wherein the at least one membrane is configured for contact with a first ultrasonic transceiver at a first distance from the first opening and a second ultrasonic transceiver at a second distance from the first opening, and for transmission of ultrasound into the measurement volume.

In some implementations, an adapter for volumetric capnography can include: a housing enclosing a measurement volume; a first tube portion including a first opening in a first end of the first tube portion, wherein a second end of the first tube portion is coupled to a first end of the housing; a second tube portion including a second opening in a first end of the second tube portion, wherein a second end of the second tube portion is coupled to a second end of the housing; and at least a first IR transparent window and a second IR transparent window in the housing, providing a line of sight through the measurement volume; wherein the adapter includes at least one membrane attached to the housing, wherein the at least one membrane separates the measurement volume from an outside of the adapter, and wherein the at least one membrane is configured for contact with a first ultrasonic transceiver at a first distance from the first opening and a second ultrasonic transceiver at a second distance from the first opening, and for transmission of ultrasound into the measurement volume.

In some implementations, the line of sight is at a third distance from the first opening, positioned between the first distance and the second distance. In some implementations, the at least one membrane is at least partly included of a different material than the housing. In some implementations, the at least one membrane includes a metal layer. In some implementations, a surface of the membrane facing the housing is included of a same material as the housing. In some implementations, the at least one membrane includes a thickness of no more than 100 micrometers. In some implementations, the at least one membrane includes a thickness of no more than 50 micrometers. In some implementations, the at least one membrane includes a thickness of no more than 20 micrometers.

In some implementations, the at least one membrane includes a first membrane at the first distance and a second membrane at the second distance. In some implementations, the housing includes a planar wall opposite to the first membrane and the second membrane, wherein the first membrane is planar and a normal vector to a center of an inside surface of the first membrane is directed towards a reflection spot on the planar wall, wherein the second membrane is planar and a normal vector to the center the inside surface of the second membrane is directed towards the reflection spot. In some implementations, the line of sight is parallel to the planar wall.

In some implementations, at least one of the first membrane is circular around a center of the first membrane and the second membrane is circular around the center of the second membrane. In some implementations, the at least one membrane is welded to the housing. In some implementations, the adapter includes an RFID tag including unique identification data. In some implementations, the first tube portion is configured to be attached to a tube inserted into a respiratory tract of a patient and the second tube portion is configured to be connected to a tube connected to a ventilator.

### BRIEF DESCRIPTION OF THE DRAWINGS

Various implementations will be described hereinafter with reference to the accompanying drawings. These implementations are illustrated and described by example only and are not intended to limit the scope of the disclosure. In the drawings, similar elements have similar reference numerals. It is to be understood that the accompanying drawings, which are incorporated in and constitute a part of this specification, are for the purpose of illustrating concepts disclosed herein and may not be to scale.

FIG. 1 is a schematic diagram of a patient in which an adapter, according to various implementations of the present disclosure, is coupled between a tube inserted into the respiratory tract of the patient and a tube coupled to a ventilator.

FIG. 2 is a schematic profile view of an adapter, according to various implementations of the present disclosure.

FIG. 3 is a schematic side cross-sectional view of the adapter of FIG. 2, according to various implementations of the present disclosure.

FIG. 4 is a schematic side cross-sectional view of the adapter of FIG. 2 with a sensor device coupled to the adapter, according to various implementations of the present disclosure.

FIG. 5 is a schematic plan view of the sensor device shown in FIG. 4, according to various implementations of the present disclosure.

FIG. 6 is a schematic perspective view of a system comprising an adapter, according to the implementations, of FIGS. 1-4 and a sensor device coupled to the adapter, according to various implementations of the present disclosure.

FIG. 7 is a schematic cross-sectional view of an adapter, according to various implementations of the present disclosure.

FIG. 8 is a schematic cross-sectional of an adapter, according to various implementations of the present disclosure.

FIG. 9 is a schematic cross-sectional view of a configuration of one or more membranes, according to various implementations of the present disclosure.

FIG. 10 is a schematic side cross-sectional view of a configuration of an ultrasonic transceivers, according to various implementations of the present disclosure.

### DETAILED DESCRIPTION

Although several implementations, examples, and illustrations are disclosed below, it will be understood by those of ordinary skill in the art that the devices, systems, and methods described herein extend beyond the specifically disclosed implementations, examples, and illustrations and includes other uses of the devices, systems, and methods and obvious modifications and equivalents thereof. Implementations are described with reference to the accompanying figures, wherein like numerals refer to like elements throughout. The terminology used in the description presented herein is not intended to be interpreted in any limited or restrictive manner simply because it is being used in conjunction with a detailed description of some specific implementations of the devices, systems, and methods. In addition, implementations can comprise several novel features. No single feature is solely responsible for its desirable attributes or is essential to practicing the devices, systems, and methods herein described.

The present disclosure may be understood by reference to the following detailed description. It is noted that, for purposes of illustrative clarity, certain elements in various drawings may not be drawn to scale, may be represented schematically or conceptually, or otherwise may not correspond exactly to certain physical configurations of implementations.

There are two distinct types of capnography, namely, conventional, time-based capnography and volumetric capnography. Conventional, time-based capnography allows for only qualitative and semi-quantitative, and sometimes misleading, measurements. For these reasons, volumetric capnography has emerged as the preferred method to assess the quality and quantity of ventilation. In volumetric capnography the flow of gas to and from a patient is measured simultaneously with measurement of the concentration of a tracer substance.

Described herein are systems, devices, and methods for a sensor device for volumetric capnography configured to be attached to an adapter comprising a housing enclosing a measurement volume. The sensor device can enable simultaneous measurement of the gas flow through the adapter and measurement of the concentration of a tracer substance in gas flowing through the adapter, and the sensor device can also enable the adapter to have a low resistance to the gas flow in relation to the adapters of the prior art. Furthermore, described herein is a sensor device for volumetric capnography that can be configured to be attached to an adapter comprising a housing enclosing a measurement volume. The sensor device can enable simultaneous measurement of the gas flow through the adapter and measurement of the concentration of a tracer substance in gas flowing through the adapter, and the sensor device can further enable the adapter to have a smaller dead volume than adapters according to the prior art. Lastly, also provided is a system that can comprise an adapter and a sensor device for simultaneous measurement of the flow of gas and the concentration of the tracer gas.

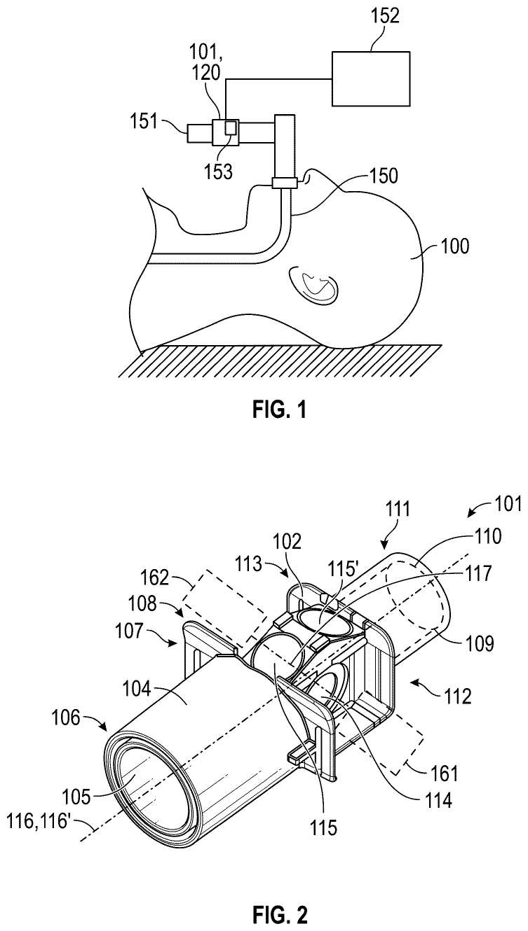

FIG. 1 illustrates schematically an adapter 101 that can be coupled (e.g., connected) between a tube 150 inserted into the respiratory tract of a patient 100 and a tube 151 coupled to a ventilator (not shown). A sensor device 120 for volumetric capnography can be coupled (e.g., connected) to the adapter 101. The sensor device 120, in some implementations, provides a solution for volumetric capnography. The sensor device 120, according to some implementations, enables volumetric capnography with a minimal dead volume and minimal additional flow resistance. The sensor device 120 can comprise a computing device 153, which determines the concentration of a tracer substance in the gas flowing through the adapter 101 and the flow rate of the gas flowing through the adapter 101 based at least on signals from the sensor device 120. Also shown in FIG. 1 is an external unit 152, to which the determined flow and concentration are transmitted. The tracer substances of interest in volumetric capnography can be, for example, carbon dioxide, ethanol, nitrous oxide, halothane, isoflurane, desflurane, sevoflurane, and/or acetone.

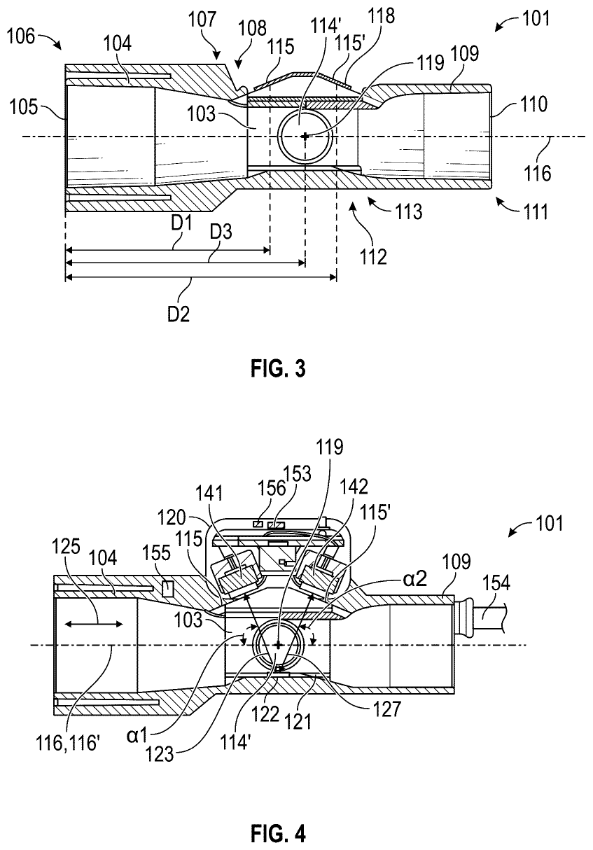

FIG. 2 illustrates a schematic perspective view of the adapter 101 for volumetric capnography, according to some implementations. FIG. 3 illustrates a schematic side cross-sectional view of the adapter 101 of FIG. 2, and FIG. 4 illustrates a schematic side cross-section of the adapter 101 of FIG. 2 with a sensor device coupled to the adapter 101. The adapter 101 can include a housing 102 enclosing a measurement volume 103 (FIGS. 3 and 4). The adapter 101 can include a first tube portion 104 comprising a first opening 105 in a first end 106 of the first tube portion 104. The adapter 101 can further include a second end 107 of the first tube portion 104 coupled (e.g., connected) to a first end 108 of the housing 102. The adapter 101 can further include a second tube portion 109 comprising a second opening 110 in a first end 111 of the second tube portion 109, wherein a second end 112 of the second tube portion 109 is coupled to a second end 113 of the housing 102.

The adapter 101 can include a first IR transparent window 114 and a second IR transparent window 114′ in the housing 102 on opposite sides of the housing 102. The first IR transparent window 114 and the second IR transparent window 114′ can provide a line of sight through the measurement volume 103. An IR light source 161 can be arranged on the outside of the first IR transparent window 114 and configured to emit infrared radiation through the first transparent window 114, through the measurement volume 103, and through the second IR transparent window 114′ (FIG. 4) in an emission direction that coincides with the line of sight. An IR light detector 162 can be arranged on the outside of the second IR transparent window 114′. The IR light detector 162 can be configured to detect light from the IR light source 161 that has passed through the measurement volume 103. The IR light source 161 and the IR light detector 162 can also include a light sensor. The IR light detector 162 can be configured to detect light at an absorption wavelength for a tracer substance to be measured. Different tracer substances can have absorption peaks at different wavelengths. A specific substance can have a number of absorption peaks. To detect a specific tracer substance, the detection wavelength can be configured to correspond to an absorption peak of the tracer substance. This can be achieved in many different ways known to skilled persons. The adapter 101 can further include a first membrane 115 attached to the housing 102 and a second membrane 115′ also attached to the housing 102. The first membrane 115 and the second membrane 115′ can separate the measurement volume 103 from the outside of the adapter 101. A length axis 116 of the adapter 101 and a line of sight 117 from the IR light source 161 to the IR light detector 162 are also shown in FIG. 2. The gas can flow through the adapter 101 along and/or substantially along the length axis 116. The line of sight 117 can be the center of a light beam from the IR light source 161 to the IR light detector 162. The length axis 116 can be made to cross the line of sight 117, by parallel translation of the length axis 116. The line of sight 117 and the length axis 116 can define a plane (e.g., a symmetry plane).

As shown in FIG. 2, the first membrane 115 and the second membrane 115′ can be circular around the center of the first membrane 115 and the second membrane 115′, but other shapes are also possible such as rectangular, quadratic, etc. The ultrasonic transceivers 141, 142, can also be circularly cylindrical, but other shapes are possible, such as rectangular, quadratic, etc.

As can be seen in FIGS. 3 and 4, the first membrane 115 can be configured for contact with a first ultrasonic transceiver 141 at a first distance D1 from the first opening 105 and a second ultrasonic transceiver 142 at a second distance D2 from the first opening 105, and for transmission of ultrasound into the measurement volume 103. The first distance D1 can be defined as the distance to the center of the first membrane 115 and the second distance D2 is defined as the distance to the center of the second membrane 115′. Even if a first membrane 115 and a second membrane 115′ are shown in FIGS. 2-4, it should be noted that it would be possible to have only one continuous membrane as is indicated by the thick line 118. The line of sight, indicated by the cross 119, can be at a third distance D3 from the first opening 105, being between the first distance D1 and the second distance D2.

As mentioned above, FIG. 4 shows a schematic side cross section view of the adapter 101 of FIG. 2 having a housing 102 enclosing the measurement volume 103 with a sensor device 120 coupled (e.g., attached, connected, etc.) to the adapter 101. The housing 102 can comprise a planar wall 121 opposite to the first membrane 115 and the second membrane 115′. The first membrane 115 can be planar and the normal vector to the center of the inside surface of the first membrane 115 can be directed towards a reflection spot 122 on the planar wall 121 as is indicated by the first arrow 123. The second membrane 115′ can be planar and the normal vector to the center of the inside surface of the second membrane 15′ is directed towards the reflection spot 122 on the planar wall 121 as is indicated by the second arrow 124. The sensor device 120 can include an ultrasonic sensor comprising a first ultrasonic transceiver 141 in contact with the first membrane 115 and a second ultrasonic transceiver 142 in contact with the second membrane 115′. A reflective surface (e.g., reflection spot 122) in the adapter 101 can allow the first ultrasonic transceiver 141 to be arranged on the same side of the adapter 101 as the second ultrasonic transceiver 142, opposite the reflective surface. The attachment of the sensor device 120 on the adapter 101 can be facilitated with such an arrangement. The flow direction can be indicated by the third arrow 125. The flow direction can vary and change direction according to the inhale and exhale of the patient 100, which inhale and exhale is controlled by a ventilator (not shown). The angle α1 between the first direction (e.g., a first main direction) and a second length axis 116′, can be equal to the angle α2 between the second direction (e.g., a second main direction) and the second length axis 116′. The second length axis 116′ can be aligned with the length axis 116. Such an arrangement of the ultrasonic transceivers 141, 142 can maximize the detection efficiency of the ultrasound. The light can be emitted from a spot arranged between the first position and the second position along the second length axis 116′. Such an arrangement of the line of sight minimizes the measurement volume 103 as the measurement volume 103 can be defined by the cross-sectional area of the measurement volume 103 and the distance between the first ultrasonic transceiver 141 and the second ultrasonic transceiver 142. The emission direction of the IR light (e.g., from the IR light source 161) can be transversal and/or perpendicular to the second length axis 116′. This is helpful in that it facilitates the manufacturing of the adapter 101 to ensure attachment of the sensor device 120 on the adapter 101.

During operation of the sensor device 120, the first ultrasonic transceiver 141 can emit first ultrasound pulses during first time periods. The first ultrasound pulses can be emitted into the measurement volume 103 through the first membrane 115 in a first direction indicated by the first arrow 123 towards the reflection spot 122. After reflection in the reflection spot 122, the ultrasound pulses travel in the second direction indicated by the second arrow 127 and are then detected by the second ultrasonic transceiver 142. First transmission times can be determined for the first ultrasound pulses as the time difference between emission of each one of the first ultrasound pulses and the reception of the corresponding first ultrasound pulse. The second ultrasonic transceiver 142 can emit second ultrasound pulses during second time periods. The second ultrasound pulses are emitted into the measurement volume 103 through the second membrane 115′ in a second direction indicated by the second arrow 127 towards the reflection spot 122. After reflection in the reflection spot 122, the ultrasound pulses can travel in the first direction indicated by the first arrow 123 and can then be detected by the first ultrasonic transceiver 141. Second transmission times can be determined for the second ultrasound pulses as the time difference between emission of each one of the first ultrasound pulses and the reception of the corresponding first ultrasound pulse.

The ultrasound pulses can be affected by the flowing gas. Ultrasound pulses that travel against the direction of the flow of gas can move slower while ultrasound pulses that travel in the same direction as the flow of gas can move faster. Thus, the transmission times can be different upstream and downstream. The difference in transmission time for an upstream pulse and a downstream pulse can depend on the flow speed of the gas and the distance travelled between the first ultrasonic transceiver 141 and the second ultrasonic transceiver 142. The flow speed can be determined from the distance travelled and difference in transmission times for ultrasound pulses in different directions. The relevant distance to consider in the determination of the flow speed can be the distance parallel to the flow direction. In the implementations of FIGS. 2-4, the ultrasound pulses travel at an angle to the flow direction. The relevant distance to consider for the determination of the flow speed can be the distance from the first ultrasonic transceiver 141 to the second ultrasonic transceiver 142. This distance can be expressed as the difference between the third distance D3 and the first distance D1, e.g., D3-D1. The measurement of the flow speed with an ultrasonic measurement and the measurement of the concentration of the tracer substance with an IR absorption measurement are both non-intrusive techniques.

As the first ultrasonic transceiver 141 and the second ultrasonic transceiver 142 are not point sources, the distance between the first ultrasonic transceiver 141 and the second ultrasonic transceiver 142 can vary depending on the points of the ultrasonic transceivers between which the distance is defined. However, if the transmission times are measured between the center of the emitted ultrasound pulse and the center of the received pulse, the transmission times can correspond to a path from the center of the first ultrasonic transceiver 141 to the center of the second ultrasonic transceiver 142.

During operation, the concentration of the gas in the measurement volume 103 can be measured using the IR light source 161 and the IR light detector 162. The concentration can be determined based on the drop in intensity detected at the IR light detector 162 and based on the distance between the first IR transparent window 114 the second IR transparent window 114′. In the implementations of FIGS. 2-4, the line of sight 117 can be parallel and/or substantially parallel to the planar wall 121.

The relevant distances can be available to a computing device 153 that determines the concentration of the tracer substance and the flow rate of the gas. In the implementations of FIG. 1, the computing device 153 can be in the control unit 152. It is, however, also possible to have the computing device 153 integrated in the sensor device 120. The sensor device 120 can comprise a cable 154 for output of data from the sensor device 120.

In the implementations of FIG. 4, the adapter 101 can include a radio-frequency identification (RFID) tag 155. The RFID tag 155 can store information such as the cross-sectional area and the distance between windows 114, 114′. The sensor device 120 of FIG. 4 can comprise an RFID reader 156. The sensor device 120 can read the information regarding the cross-sectional area and the distance between the IR transparent windows 114, 114′ from the RFID tag 155. Alternatively or additionally, the RFID tag 155 can comprise unique identification data. The unique identification data can be used to track the individual adapter 101. After an adapter 101 has been used, it can be discarded to prevent contamination of the next patient 100 by the previous patient. With a unique identification data, the adapter 101 can be controlled automatically by the computing device 153. In some implementations, the sensor device 120 does not comprise an RFID reader 156. Rather, the information on the unique identification data in this case be read by a separate RFID reader. It is of course also possible to have the unique identification data applied as a bar code or matrix code. Such a code can be read automatically by an optical reader similar to the RFID reader 156.

In some implementations, the ultrasonic transceivers 141, 142 are not in contact with the gas flowing through the adapter 101. Due in part to the membranes 115, 115′, the first ultrasonic transceiver 141 and the second ultrasonic transceiver 142 may remain isolated from the gas flowing in the adapter 101 during use. As a result, the sensor device 120 may not be in direct contact with the flow of air. Consequently, the sensor device 120 does not require thorough sterilization between users (e.g., between different patients 100). In some implementations, the adapter 101 can be exchanged between each patient 100 and is delivered sterilized in a sealed package.

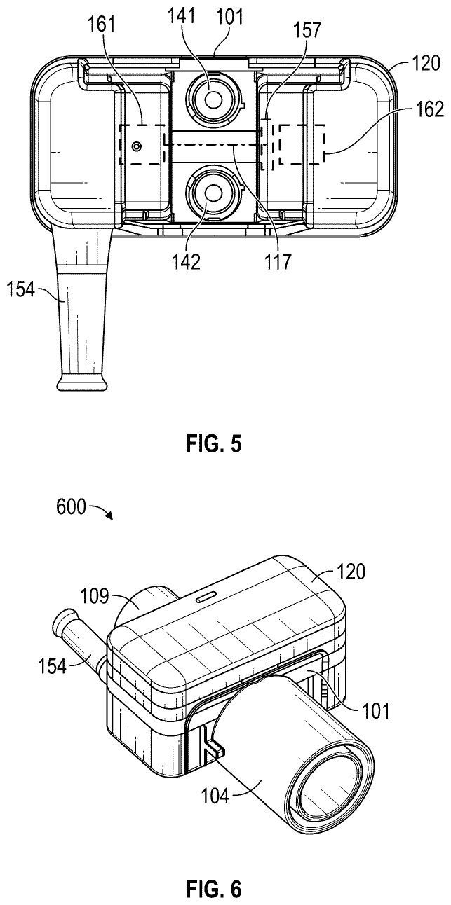

FIG. 5 illustrates a schematic plan view of the sensor device 120 shown in FIG. 4. As shown in FIG. 5, the adapter 101 can be coupled (e.g., connected) to the sensor device 120 between the IR light source 161 and the IR light detector 162. An interference filter 157 can be arranged in front of the IR light detector 162. The interference filter 157 can transmit a narrow wavelength band at the relevant absorption wavelength of the tracer substance to be detected. FIG. 6 illustrates a schematic perspective view of a system 600 comprising an adapter 101 according to the implementations of FIGS. 1-4 and a sensor device 120 coupled to the adapter. Also shown in FIG. 6 are the first tube portion 104 and the second tube portion 109 of the adapter and the cable 154 for output of data from the sensor device 120.

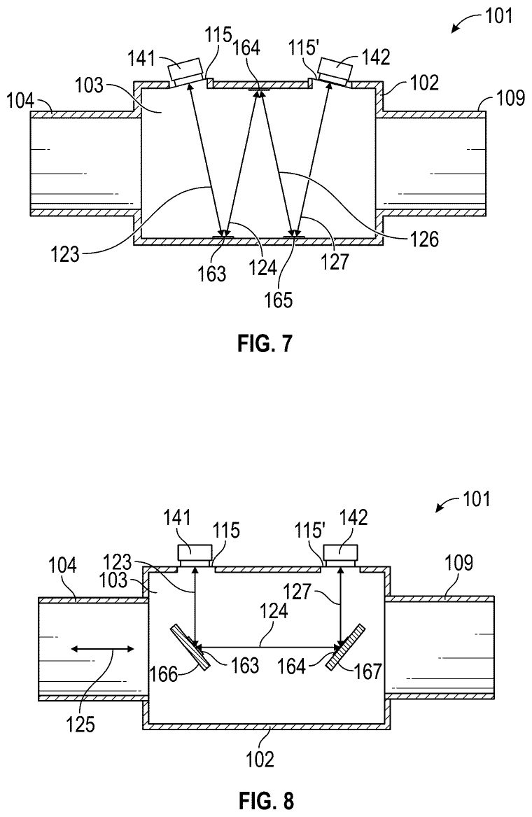

FIG. 7 illustrates a schematic side cross-sectional view of an adapter 101, according to some implementation. During first time periods (e.g., initial time periods), the first ultrasonic transceiver 141 can transmit ultrasound through the first membrane 115 along the first direction indicated by the first arrow 123. The ultrasound can then be reflected in the first reflection spot 163 on the inside of the housing 102 into the direction indicated by the second arrow 124 towards the second reflection spot 164. The ultrasound can then be reflected in the second reflection spot 164 on the inside of the housing 102 into the direction indicated by the third arrow 126 towards the third reflection spot 165. The ultrasound can then be reflected in the third reflection spot 165 on the inside of the housing 102 into the second direction indicated by the fourth arrow 127 towards the second ultrasonic transceiver 142.

During second time periods (e.g., subsequent time periods), the second ultrasonic transceiver 142 can transmit ultrasound through the second membrane 115′ along the direction indicated by the fourth arrow 127. The ultrasound can then be reflected in the third reflection spot 165 on the inside of the housing 102 into the direction indicated by the third arrow 126 towards the second reflection spot 164. The ultrasound can then be reflected in the second reflection spot 164 on the inside of the housing 102 into the direction indicated by the second arrow 124 towards the first reflection spot 163. The ultrasound can then be reflected in the first reflection spot 165 on the inside of the housing 102 into the direction indicated by the first arrow 123 towards the first ultrasonic transceiver 141.

FIG. 8 illustrates a schematic side cross-sectional view of an adapter 101, according to some implementation. During first time periods, the first ultrasonic transceiver 141 can transmit ultrasound through the first membrane 115 along the direction indicated by the first arrow 123 towards the first reflection spot 163. The ultrasound can then be reflected in the first reflection spot 163 on a first reflector 166 arranged in the in the measurement volume 103 to the direction indicated by the second arrow 124 towards the second reflection spot 164 on a second reflector 167 arranged in the in the measurement volume 103. The ultrasound can then be reflected in the second reflection spot 164 into the second direction indicated by the third arrow 127 towards the second ultrasonic transceiver 142.

During second time periods, the second ultrasonic transceiver 142 can transmit ultrasound through the second membrane 115′ along the second direction indicated by the third arrow 127 towards the second reflection spot 164. The ultrasound can then be reflected in the second reflection spot 164 into the direction indicated by the second arrow 124 towards the first reflection spot 163. The ultrasound can then be reflected in the first reflection spot 163 into the direction indicated by the first arrow 123 towards the first ultrasonic transceiver 141.

In some implementations, the first ultrasonic transceiver 141 and the second ultrasonic transceiver 142 are arranged on opposite sides of the housing 102. The connection of the sensor device 120 on the adapter 101 can be facilitated by arranging the first ultrasonic transceiver 141 and the second ultrasonic transceiver 142 as shown in FIGS. 4-7 as the sensor device 120 can be configured in a U-shape.

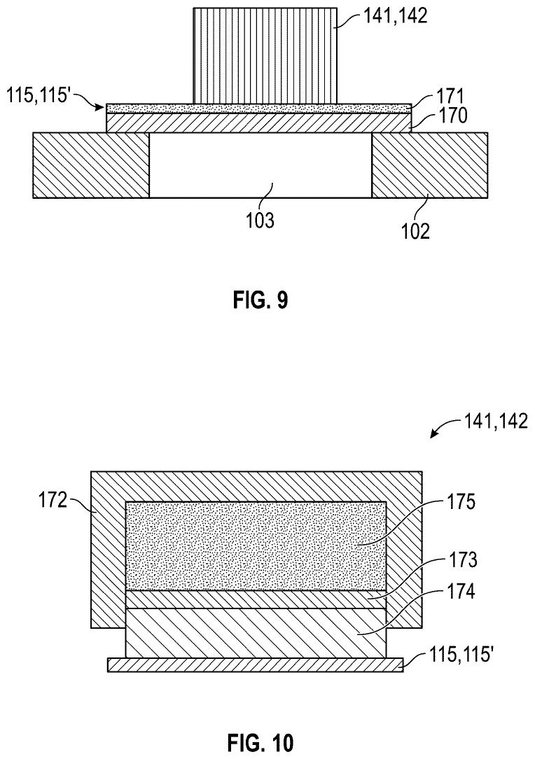

FIG. 9 illustrates a schematic side cross-sectional view of the arrangement of one of the membranes 115, 115′ according to some implementations, on the housing 102. At least one of the membranes 115, 115′ can have the illustrated configuration. Part of the wall of the housing 102 and an ultrasonic transceiver 141, 142, is illustrated in FIG. 9. The membrane 115, 115′ can be comprised of two different materials. For example, the first layer 170 of the membrane 115, 115′, can be comprised of the same material as the housing 102. The first layer 170 can face the housing 102. The material of the housing 102 can be a polymer, which allows the housing 102 to be injection molded. The polymer can be flexible and/or sufficiently flexible to allow the first layer 170 to spring back after having been pushed down by the ultrasonic transceiver 141, 142. By having the first layer 170 of the membrane 115, 115′ be of the same material as the housing 102, it is possible to weld the membrane 115, 115′ to the housing 102 for coupling (e.g., connecting) polymer components. In some implementations, the membranes 115, 115′ are attached using an adhesive. The membrane 115, 115′, can also include a second layer 171 comprising of metal. The metal can comprise aluminium. A benefit of a metal second layer 171 is that it ensures that the membrane 115, 115′ can be gas tight. If the metal second layer 171 is thin and/or sufficiently thin, the metal second layer 171 does not affect the flexibility of the polymer first layer 170. The membrane 115, 115′ can have a thickness of at least no more than approximately 100 micrometres, no more than approximately 50 micrometres, and/or no more than approximately 20 micrometres. The aluminium metal second layer 171 can be no more than half of the total thickness. As mentioned above, the membrane 115, 115′ could be manufactured as a part of the housing 102, e.g., by making the part of the housing 102 constituting the membranes 115, 115′ sufficiently thin during injection moulding. By having the membranes 115, 115′ as separate components, the fabrication of the adapter 101 can be facilitated. By preheating the membranes 115, 115′ before attachment of the membranes 115, 115′ to the housing 102, the membranes 115, 115′ can be pre-tensioned.

FIG. 10 illustrates a schematic cross-sectional view of the configuration of one of the ultrasonic transceivers 141, 142. The first ultrasonic transceiver 141, as well as the second ultrasonic transceiver 142, can be circularly cylindrical and/or substantially circularly cylindrical as is illustrated in FIGS. 4 and 5. The ultrasonic transceiver 141, 142, according to the implementations of FIG. 10, can comprise a frame 172 comprising polymer, rubber, and/or metal or any other suitable material. The ultrasonic transceiver 141, 142, can include a piezoelectric disk 173 (e.g., a piezoelectric crystal) which can be connected to the computing device 153 mentioned above. The ultrasonic transceivers 141, 142, can be controlled by the computing device 153 in such a way that they are either in transmitting mode or receiving mode.

The ultrasonic transceiver 141, 142 can also comprise an acoustic impedance matching layer 174 having an inner side in contact with the piezoelectric disk 173 and an outer side being configured to be in contact with the membrane 115, 115′, of the adapter 101. The ultrasonic transceiver 141, 142, can also comprise a backing layer 175 in contact with the piezoelectric disk 173, wherein the piezoelectric disk 173, can be arranged between the acoustic impedance matching layer 174 and the backing layer 175. The acoustic impedance matching layer 174 can transfer the acoustic wave in the form of the ultrasound from the piezoelectric disk 173 to the membrane 115, 115′ and from there to the air in the measurement volume 103. To maximize the energy transfer, an acoustic impedance from the piezoelectric disk 173 to the air in the measurement volume 103 can gradually increase. According to some implementations, the acoustic impedance matching layer 174 can comprise a porous material and can comprise an organic binder and/or silicon dioxide powder. The porosity of the material can comprise air bubbles in the organic binder. The backing layer 175 can comprise a resilient layer in contact with the piezoelectric disk 173 and can be a polymer foam or similar. The function of the backing layer 175 can be to provide a suitable pressure of the ultrasonic transceiver 141, 142 on the membrane 115, 115′ and also to attenuate the acoustic wave emitted from the piezoelectric disk 173 from the side facing away from the membrane 115, 115′. In this way, disturbances from the acoustic wave from the side facing away from the membrane 115, 115′ are avoided.

The person skilled in the art realizes that the present disclosure is not limited to the implementations described above. The person skilled in the art further realizes that modifications and variations are possible within the scope of the appended claims. Additionally, variations to the disclosed implementations can be understood and effected by the skilled person in practicing the claimed disclosure, from studying the drawings, the disclosure and the appended claims.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number, respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments.

While certain embodiments have been described, these embodiments have been presented by way of example only, and are not intended to limit the scope of the disclosure. For example, while illustrated embodiments include preparation for direct hybrid bonding, the skilled artisan will appreciate that the techniques taught herein can be useful for direct metal bonding even in the absence of direct dielectric bonding. Indeed, the novel apparatus, methods, and systems described herein may be embodied in a variety of other forms; furthermore, various omissions, substitutions and changes in the form of the methods and systems described herein may be made without departing from the spirit of the disclosure. For example, while blocks are presented in a given arrangement, alternative embodiments may perform similar functionalities with different components and/or circuit topologies, and some blocks may be deleted, moved, added, subdivided, combined, and/or modified. Each of these blocks may be implemented in a variety of different ways. Any suitable combination of the elements and acts of the various embodiments described above can be combined to provide further embodiments. It is also contemplated that various combinations or sub-combinations of the specific features and aspects of the embodiments may be made and still fall within the scope. The accompanying claims and their equivalents are intended to cover such forms or modifications as would fall within the scope and spirit of the disclosure.

What is claimed is:

1. A sensor device for volumetric capnography configured to be attached to an adapter, the sensor device comprising:
a housing enclosing a measurement volume, wherein the adapter is configured for gas flow along a first length axis through the measurement volume, and wherein a second length axis of the sensor device is aligned with the first length axis;
an infrared (IR) light source and an IR light detector, wherein the IR light source and the IR light detector are positioned on opposite sides of the housing, the IR light source configured to emit IR light in an emission direction towards the IR light detector, wherein the emission direction is at an angle to the second length axis, and wherein the second length axis and the emission direction define a symmetry plane;
a first ultrasonic transceiver configured to contact the adapter and to emit an ultrasound centered at a first position along the second length axis, the ultrasound directed at least partly in a first direction along the second length axis and at least partly transversally to the second length axis; and
a second ultrasonic transceiver configured to contact the adapter and to emit ultrasound centered at a second position along the second length axis, the ultrasound being directed at least partly in a second direction opposite to the first direction along the second length axis and at least partly transversal to the second length axis.

2. The sensor device of claim 1, wherein the first ultrasonic transceiver is configured to detect ultrasound that has been emitted from the second ultrasonic transceiver, and the second ultrasonic transceiver is configured to detect ultrasound that has been emitted from the first ultrasonic transceiver.

3. The sensor device claim 1, wherein the first ultrasonic transceiver and the second ultrasound transceiver are both arranged on a same side of the symmetry plane, and wherein the first ultrasonic transceiver is configured to detect ultrasound from the second ultrasonic transceiver, which ultrasound has travelled through the measurement volume and has been reflected in the adapter.

4. The sensor device of claim 3, wherein the first ultrasonic transceiver is configured to emit ultrasound in a first main direction at a first angle (α1) to the second length axis, and the second ultrasonic transceiver is configured to emit ultrasound in a second main direction at a second angle (α2) to the second length axis, wherein a size of the first angle (α1) is equal to a size of the second angle (α2).

5. The sensor device of claim 1, wherein the IR light is emitted from a spot arranged between the first position and the second position along the second length axis.

6. The sensor device of claim 1, wherein the emission direction of the IR light is perpendicular to the second length axis.

7. The sensor device of claim 1, wherein at least one of the first ultrasonic transceiver and the second ultrasonic transceiver comprises:
an acoustic impedance matching layer having an inner side and an outer side configured to be in contact with the adapter;
a piezoelectric disk attached in contact with the inner side of the acoustic impedance matching layer; and
a backing layer in contact with the piezoelectric disk, wherein the piezoelectric disk is positioned between the acoustic impedance matching layer and the backing layer.

8. The sensor device of claim 7, wherein the acoustic impedance matching layer comprises a porous material.

9. The sensor device of claim 7, wherein the backing layer comprises a resilient layer in contact with the piezoelectric disk.

10. The sensor device of claim 1, wherein the adapter comprises:
a first tube portion comprising a first opening in a first end of the first tube portion, wherein a second end of the first tube portion is coupled to a first end of the housing;
a second tube portion comprising a second opening in a first end of the second tube portion, wherein a second end of the second tube portion is coupled to a second end of the housing; and
at least a first transparent window and a second transparent window in the housing, the first transparent window and the second transparent window providing a line of sight through the measurement volume;
wherein the adapter comprises at least one membrane attached to the housing, wherein the at least one membrane separates the measurement volume from an outside of the adapter, wherein the at least one membrane is configured for contact with a first ultrasonic transceiver at a first distance from the first opening and a second ultrasonic transceiver at a second distance from the first opening, and for transmission of ultrasound into the measurement volume.

11. An adapter for volumetric capnography, the adapter comprising:
a housing enclosing a measurement volume;
a first tube portion comprising a first opening in a first end of the first tube portion, wherein a second end of the first tube portion is coupled to a first end of the housing;
a second tube portion comprising a second opening in a first end of the second tube portion, wherein a second end of the second tube portion is coupled to a second end of the housing; and
at least a first IR transparent window and a second IR transparent window in the housing, providing a line of sight through the measurement volume;
wherein the adapter comprises at least one membrane attached to the housing, wherein the at least one membrane separates the measurement volume from an outside of the adapter, and wherein the at least one membrane is configured for contact with a first ultrasonic transceiver at a first distance from the first opening and a second ultrasonic transceiver at a second distance from the first opening, and for transmission of ultrasound into the measurement volume.

12. The adapter of claim 11, wherein the line of sight is at a third distance from the first opening, positioned between the first distance and the second distance.

13. The adapter of claim 11, wherein the at least one membrane is at least partly comprised of a different material than the housing.

14. The adapter of claim 11, wherein the at least one membrane comprises a first membrane at the first distance and a second membrane at the second distance.

15. The adapter of claim 14, wherein the housing comprises a planar wall opposite to the first membrane and the second membrane, wherein the first membrane is planar and a normal vector to a center of an inside surface of the first membrane is directed towards a reflection spot on the planar wall, wherein the second membrane is planar and a normal vector to the center the inside surface of the second membrane is directed towards the reflection spot.

16. The adapter of claim 15, wherein the line of sight is parallel to the planar wall.

17. The adapter of claim 14, wherein at least one of the first membrane is circular around a center of the first membrane and the second membrane is circular around the center of the second membrane.

18. The adapter of claim 11, wherein the at least one membrane is welded to the housing.

19. The adapter of claim 11, further comprising an RFID tag comprising unique identification data.

20. The adapter of claim 11, wherein the first tube portion is configured to be attached to a tube inserted into a respiratory tract of a patient and the second tube portion is configured to be connected to a tube connected to a ventilator.
